(19) United States
(12) Patent Application Publication
(10) Pub. No.: US 2002/0019021 A1
(43) Pub. Date: Feb. 14, 2002

(54) METHOD FOR DETERMINING THE ANTICOAGULATORY POTENTIAL OF A SAMPLE

(76) Inventor: MICHAEL KRAUS, MARBURG (DE)

Correspondence Address: FINNEGAN HENDERSON FARABOW, GARRETT AND DUNNER, FRANKLIN SQUARE BLDG SUITE 700, 1300 I STREET N W, WASHINGTON, DC, 20005

(21) Appl. No.: 09/187,035

(22) Filed: Nov. 6, 1998

(30) Foreign Application Priority Data
Nov. 7, 1997 (DE) 197 49 197.9 1

(51) Int. Cl. C12Q 1/56; C12N 9/74; C08B 37/10; C07K 14/00; C07K 16/00

(52) U.S. Cl. 435/13; 435/214; 530/381; 530/384; 536/21

(57) ABSTRACT

The application relates to a method for determining the anticoagulatory potential of a sample by adding thrombomodulin and thromboplastin in a coagulation test.

The application relates to a method for determining the anticoagulatory potential of a sample by adding thrombomodulin and thromboplastin in a coagulation test.

The activation of coagulation leads to the conversion of the proenzyme prothrombin into the active protease thrombin. Thrombin accelerates its formation itself in that it activates the cofactors factor V and factor VIII by means of proteolytic cleavage. Together with the proteases factor Xa and IXa, respectively, these activated cofactors form active enzyme/cofactor complexes on phospholipid surfaces, the activity of which complexes is higher than that of the proteases on their own by a factor of about 10,000. This positive feedback results in large quantities of thrombin being formed in an almost explosive manner. Thrombin converts fibrinogen into fibrin, which normally leads to wound closure and wound healing. In order to prevent a life-threatening extension of the coagulation, which would lead to occlusion of the vascular system of the body, that is to thromboses, both the active protease and further activation proteases have to be inhibited. In the body, active proteases are neutralized by protease inhibitors by means of forming covalent complexes. The most important protease inhibitor is antithrombin III, whose anticoagulatory effect is accelerated by heparin sulfates. The continued formation of active coagulation proteases is interrupted by thrombin itself, acting through a feedback mechanism. Thrombin binds to the membrane protein thrombomodulin and thereby loses its procoagulatory properties such as the activation of platelets or the conversion of fibrinogen. In the presence of calcium ions, the thrombin/thrombomodulin complex converts the proenzyme protein C into the active protease protein Ca (APC) (effect A). In addition, thrombomodulin itself exerts an anticoagulatory effect through its glycosylation, a heparan sulfate. This increases the rate at which an inactive thrombin/antithrombin III complex is formed (Dittmann W A, Majerus P W, Blood 1990; 75: 329-336, Bourin M-C, Lindahl U, Biochem J 1990; 270: 419-425). Together with its cofactor protein S, the APC which is produced forms a complex which proteolytically cleaves, and thereby inactivates, the active cofactors factor VIIIa and factor Va. APC thereby interrupts the strong stimulation by these cofactors and the further formation of factors Xa and thrombin. Another membrane protein, i.e. the endothelial protein C receptor, appears to stimulate the protein C-activating activity of the thrombin/thrombomodulin complex.

This protein C system, which is described above, constitutes an important anticoagulatory mechanism. This is confirmed by the fact that persons with hereditary or acquired deficiencies or defects in protein C or protein S are highly likely to suffer from thromboses, in particular recurring venous thromboses. Other factors besides protein C and protein S can influence the activity of the system, for example von Willebrand factor and factor IXa, which are able to protect factor VIIIa from proteolytic degradation. Acquired disturbances can also have their origin in the formation of lupus anticoagulants. These are antibodies which are directed against phospholipids and which interfere with the binding, which is necessary for proper function, of the protease/cofactor complexes to phospholipid surfaces. A mutant of factor V which can no longer, or at least only very poorly, be inactivated by APC has also been described. Mutations of the factors involved in the thrombin/thrombomodulin complex, and which lead to a reduced formation of activated protein C, such as mutations of thrombomodulin itself, of protein C and of thrombin, are also known.

Defects or deficiencies of antithrombin III are another important cause of the formation of thromboses. Commonly, antithrombin III is determined by adding thrombin and heparin to a highly dilute sample and determining the residual thrombin by adding a chromogenic substrate or fibrinogen and determining the transformation rate or the formation of a fibrin clot.

Because of the many possible disturbances of the protein C system, it makes sense in clinical diagnosis to use a screening test which generally indicates a disturbance in this system, i.e. a disturbance of its anticoagulatory potential. This is particularly the case when specific disturbances, such as, in this case, due to von Willebrand factor, factor IXa, lupus anticoagulant or the mutation of factor V, can only be analyzed in a very elaborate manner in laboratories which are specially experienced in the area. In addition to this, a screening test for determining the potential of the protein C system can also indicate disturbances whose causes, such as, for example, the influence of acute phase reactions or inflammations, can only be poorly clarified in detail since it is not possible to establish conclusively the interaction of different factors from a total of individual factor determinations. Furthermore, such a screening test can concomitantly detect disturbances whose causes are at present still unknown. Such a test can therefore be used to search, in a patient, for individual or multiple factor disturbances which can lead to an increased risk of thrombosis.

The use of a test which determines the anticoagulatory potential of a sample, that is of the protein C system and/or of antithrombin III, goes beyond the determination of an individual cause and achieves a value of its own which makes its mark in clinical practice for recognizing an increased tendency to thrombosis (thrombophilia) and, as a result, consequences for therapy, such as anticoagulation therapy using coumarin derivatives or heparins. The monitoring and control of anticoagulation therapy is consequently an additional application of this test.

Until now, functionality investigations have been carried out on protein C or protein S as individual factors. For this, the sample, or protein C which has been isolated from the sample, is initially added in substoichiometric quantity to a protein C-deficient plasma. The protein C is then activated either by adding thrombin or a combination of thrombin and thrombomodulin or by adding an Agklstrodon contortrix snake venom, which is known under its trade name of Protac® (from Pentapharm, Basel, Switzerland). The protein C which is present in the sample is detected either on the basis of the increase in the coagulation time, due to the anticoagulatory effect of the protein C which is present in the sample, or by means of the transformation of a substrate which is specific for thrombin. Alternatively, the protein C activity can also be determined chromogenically in a direct manner, following activation with thrombin or Protac®, by using a substrate which is specific for APC.

The protein S determinations are carried out by mixing the sample with PS-deficient plasma. The stimulatory effect protein S on APC is measured by determining the increase in coagulation time. The APC which is required for this purpose is either added or else the protein C which is present in the PS-deficient plasma is activated with Protac® (Bertina, R M, Res Clin Lab 1990; 20: 127-138). Matschiner (U.S. Pat. No. 5,525,478) has described a method for determining protein S using thrombomodulin (see below).

Known methods for determining protein C using thrombomodulin are based on isolating protein C from the sample by means of adsorption. This protein C, which has been isolated from the sample, is then activated with thrombin/thrombomodulin complex, and the active protein C which has been generated is detected in the chromogenic test (Thiel, W. et al., Blut 1986; 52: 169-177). This method is complicated and does not determine the entire potential of the protein C system. In addition, its use is restricted to chromogenic methods, i.e. it is not possible in this way to determine the physiological repercussions on the formation of a fibrin clot.

EP 0 711 838 describes a method for functionally determining variants of factor V whose activated forms are inactivated to a lesser extent by APC than is normal(wild type)factor Va. For this determination, the sample is mixed with a factor V-deficient plasma in order to exclude interfering influences, for example factor deficiencies, lupus anticoagulants or therapeutic influences (oral anticoagulation or heparin), and a coagulation test is then carried out in the presence of activated protein C.

It has also already been reported that a method originally used for detecting thrombomodulin has been employed for detecting thrombin mutants whose characteristic feature is that they do not form any active complex with thrombomodulin. For this, the sample is diluted such that no clots, which would interfere with the subsequent determination, are formed after the prothrombin has been activated to form thrombin using enzymes, which are known per se to the skilled person, from snake venoms. After thrombomodulin and protein C have been added, the formation of activated protein C is monitored by the transformation of a chromogenic protein C substrate.

The methods which have been cited thus far are only suitable for detecting disturbances of the protein C system caused by the factor which is in each case investigated individually. For the reasons given above, they are not suitable as screening tests. The fact that the determination methods were insufficiently practicable has also so far stood in the way of introducing them as screening tests over a broad front.

In order to determine disturbances in F V, Amer et al. (Thromb. Res. 1990; 57: 247-258) modified the activated partial thromboplastin time (APTT). The APTT is a standard method for detecting disturbances in coagulation, i.e. it is used for diagnosing hemorraghic tendencies. After the sample plasma has been activated with an activating surface, coagulation is not started, as is customary in the case of APTT, by adding a solution of calcium chloride; instead, APC is added concomitantly with the calcium ions. The anticoagulatory effect of the exogenously added APC prolongs the coagulation times. Consequently, this test already recognizes many disturbances of the protein C system apart from defects or deficiencies in the protein C in the sample, since APC is added exogenously, and also disturbances which relate, for example, to the interaction of protein C and/or thrombin with thrombomodulin, since thrombomodulin is not present.

DE 44 27 785 describes a method for determining disturbances of the protein C system in which the protein C of the sample to be investigated (endogenous protein C) is first of all preactivated using a protein C activator. The effect of the resulting APC in retarding thrombin formation is then examined in a coagulation test. Known activators, such as snake venom enzymes (for example from Agkistrodon contortrix, tradename Protac®), or thrombin/thrombomodulin complexes are used as protein C activators. Formation of thrombin can be detected by way of clot formation (classical method) or using a chromogenic substrate. The coagulation tests which are used as the basis for determining the anticoagulatory effect of the protein C system comprise all the methods which are known per se to the skilled person, such as the APTT, the thromboplastin time (PT), the Russell's viper venom time (RVVT), or the addition of activated coagulation factors or of snake venoms, or enzymes from these venoms, which in the end lead to the formation of thrombin and thereby to the formation of activated factor V. As compared with previous methods, this method encompasses all disturbances of the protein C system with the exception of variants of thrombin and of thrombomodulin. When preformed thrombin/thrombomodulin complexes are used, this method can detect additionally variants of protein C, whose binding to or activation in the thrombin/thrombomodulin complex is disturbed.

FR 2 689 640 describes a method which is based on the thromboplastin time, a standard method in coagulation diagnostics, and in which coagulation is activated in a sample by adding thromboplastin and calcium. The resulting thrombin activates the protein C in the sample (endogenous protein C) when thrombomodulin is added concomitantly. The APC counteracts the formation of thrombin to an extent which depends on the efficiency with which the protein C system is functioning. After 15 minutes, further coagulation activity is interrupted by complexing the calcium ions and the thrombin which has been formed is determined by the transformation of a specific, chromogenic substrate. The quantity of thrombin which has been formed is indirectly proportional to the operability of the protein C system. All disturbances of the protein C system in the sample can be detected since both the endogenous protein C and the endogenous prothrombin are activated. However, the test suffers from some disadvantages. In the first place, this method is unsuitable for routine use as a screening test because of the long total measuring time of 16 minutes. In the second place, a clot is produced in the sample before activated protein C is actually formed, as a result of which it is only possible to use this method in combination with chromogenic measurement methods which detect the conversion of the thrombin which has been produced. As a consequence, it is no longer possible to use the traditional measurement methodology, which detects the formation of the fibrin clot. In the method developed by Duchemin et al., the fibrinogen in the sample is therefore removed, for example by adding fibrin-cleaving enzymes, prior to the investigation, in order to avoid interferences due to the resulting clot.

Similar methods are described by Rijkers et al. (Rijkers DTS et al., Thromb Haemost 1997; Supplement; 550 Abstract PS-2251) and in U.S. Pat. No. 5,051,357.

These previously described methods for activating the protein C in the sample using the endogenous prothrombin in the sample and exogenously added thrombomodulin are characterized by the following features

1. a preincubation is required in order to form activated protein C;

2. activation of the endogenous thrombin leads to the premature formation of a fibrin clot, for which reason fibrinogen in the sample has to be destroyed prior to the analysis, and therefore

3. it is only possible to use chromogenic detection methods;

4. this results, all in all, in a long period of measurement (greater than 10 minutes), due to the incubation times and/or the pretreatment of the sample.

However, the methods which would be advantageous for analyzing the potential of the protein C system would be those which permit a routine determination on current coagulometers, i.e. which make it possible to use short measurement times (less than 10 minutes) and to carry out the traditional determination of a fibrin clot.

An object of the invention was, therefore, to find a method which also makes it possible to determine the potential of the protein C system using traditional methods and short measurement times. Another object was that such a test should concomitantly detect deficits in antithrombin III.

In U.S. Pat. No. 5,525,478, Matschiner describes a method for determining the protein C potential; in this method the sample is incubated with a contact phase activator, and coagulation is then activated with a mixture of calcium chloride and thrombomodulin instead of with calcium chloride alone. Matschiner states that the coagulation time in the APTT is prolonged from 36 to 156 s when 1 U of (rabbit) thrombomodulin/ml is added. In addition, he describes methods, derived from this, for determining protein C and protein S by mixing the sample with protein C-deficient plasma or protein S-deficient plasma before using it in this test. Our own experiments (see Example 3) confirmed that it is necessary to add 5 μg of (rabbit) thrombomodulin/ml (based on the total test assay) in order to prolong the coagulation time in the APTT from approx. 30 to approx. 160 s.

Analogous determinations have also been carried out using recombinant thrombomodulin; as was to be expected, the coagulation time was found to be prolonged (Ohishi et al., Thromb. Haemostas 1993; 70: 423-426). Interestingly, however, this effect is only very weak (approx. 150 sec at approx. 1 μg of thrombomodulin/ml in the plasma; approx. 1 U of thrombomodulin/ml). However, this effect is only apparent when, as in this case, the coagulation time without adding thrombomodulin is very long (450 s). In association with these long coagulation times, retardations in the formation of thrombin have a disproportionate effect on clot formation, for which reason this prolongation by 150 s cannot be compared with the prolongation, described by Matschiner, which occurs in association with a very much shorter basic coagulation time without thrombomodulin. This long coagulation time was obtained by using a thromboplastin reagent which was very highly diluted with calcium chloride. These long coagulation times (greater than 300 s) are viewed very critically by the skilled person since the precision of the determination is very inexact. Small fluctuations in coagulation factors, in particular cofactors V and VIII, lead to disproportionately large increases in the coagulation times. Furthermore, these long measurement times are impracticable for routine determinations since they reduce the sample throughput under routine conditions.

If a normal PT is used instead of a highly diluted PT, it is only possible to demonstrate the anticoagulatory effect of thrombomodulin by using very high quantities. This is evident from the investigations carried out by Takahashi et al. (Throinb Haemostas 1995; 73: 805-811). The authors added thrombomodulin which had been concentrated from urine to normal plasma and carried out a normal PT with coagulation times without thrombomodulin of approx. 13 sec. While the coagulation time was prolonged by adding very high concentrations, the prolongation in the coagulation time was only about 17 sec. even at 1000 U/ml. This is inadequate for discriminating between normal persons and patients suffering from deficiencies in the protein C system.

Surprisingly, it was found that thromboplastin from which the heparin-like glycosylation had been removed using chondroitinase ABC does not exhibit any such pronounced prolongation of the coagulation time in the method described by Matschiner.

Recombinantly prepared thrombomodulin also lacks the glycosylation which is appropriate for increasing the rate at which thrombin is inactivated by antithrombin III (effect B), i.e. it only has the property of activating protein C as a cofactor for thrombin (effect A).

Consequently, the present invention was based on the object of providing a screening method for determining anticoagulatory potential. Anticoagulatory potential is understood as being the property of plasma to bring about a prolongation in coagulation time due to direct inhibition of thrombin and/or retardation of the formation of thrombin in a coagulation test which is based on thrombin formation.

Screening methods make special demands. Since they are intended for working through a large number of samples in a short time and, despite that, very reliably, they should not exceed a measuring time of 5 min and it should advantageously be possible to carry them out as a 1-step assay. A 1-step assay is understood as being a test in which there is no need for any preincubation times between reagent additions. For the purpose of screening relatively large groups of people, it is advantageous to use reagents which can be produced as reproducible as possible, for example to use recombinant proteins, for example in the present case to use recombinant thrombomodulin. A screening method must therefore also be operable with such a recombinant thromboplastin whatever its origin. This object was achieved by the embodiments presented in the claims.

The invention relates to a method for determining and diagnosing the anticoagulatory potential of a sample in the presence of exogenously added thrombomodulin, which method includes the following steps:

a) the following reagents are added to the sample, preferably a plasma sample:

i) exogenous thrombomodulin which can form a complex with thrombin, with this complex being able to activate the protein C in the sample, and with it being possible for the protein C to be endogenous protein C or exogenously added protein C,

ii) an activator which leads, without any further intermediate incubation, to the activation of prothrombin to form thrombin, with it being possible for the prothrombin to be endogenous prothrombin or exogenously added prothrombin,

iii) phospholipids,

iv) calcium ions,

v) and also other additional reagents which are used generally for optimizing coagulation tests,

b) the reaction is started by adding the prothrombin activator-containing reagent, and

c) the formation of thrombin is determined by measuring the transformation rate of a thrombin substrate, with this transformation rate being determined by measuring the time until a fibrin clot has formed or by the transformation rate of a labeled thrombin substrate.

Whole blood from veins or capillaries and plasma, preferably citrate plasma, may be used as a sample.

The following may preferably be used as prothrombin activators which lead, without any further intermediate incubation, to the activation of prothrombin to form thrombin: factor Xa or Va or factor Xa/Va complexes, or prothrombin activators from snake venoms, for example ecarin or textarin (Rosing J, Tans G, Thromb. Haemostas 1991; 65: 627-630) which are known per se to the skilled person, or factor X activators, such as factor IXa, VIIIa or factor IXa/VIIIa complexes, or factor X and/or factor V activators from snake venoms, for example from Russell's viper venom, which are known per se to the skilled person, preferably, however, by adding a thromboplastin-containing reagent, for example from rabbit brain or lung, or from human placenta, such as Thromborel S (from Behring Diagnostics), or of recombinant origin, such as Innovin (from Dade) or Thromborel R (from Behring Diagnostics).

The added phospholipids can be of natural or synthetic origin, preferably from tissue extracts of placenta, lung, brain or thrombocytes of human or animal origin; extracts from plants, such as soybeans, are also preferred. Advantageously, the phospholipids are added in such a quantity that a concentration of from 0.001% to 1.0% (w/v), preferably of from 0.005% to 0.5%, particularly preferably of from 0.015% to 0.15%, is obtained in the test assay.

The novel method can also be used for selectively determining defects in special coagulation factors. For this, a solution which contains the coagulation factors which are not to be codetected in the test is added to the sample employed, preferably before the sample is used in the test.

Coagulation factors which are of particular interest are, for example, AT III, protein S, protein C, factor V and prothrombin, or their variants.

In order to eliminate inferences due to heparin, the heparin which is present in the sample can be degraded or neutralized, for example using heparinase or amiries, such as polylysine, hexadimethrine, spermine, spermidine or protamine sulfate, or in an excess which is as large as possible compared with the heparin concentrations to be expected, preferably from 0.1 to 10 U/ml of test assay, particularly preferably 0.3-3 U/ml, very particularly preferably 0.7 U/ml, can be added.

It was concluded from investigations carried out into the effect of different thrombomodulins that the inhibition of thrombin is not only one of several properties of thrombomodulin but also a prerequisite for anticoagulatory activity by way of the protein C system. This finding is novel. It was furthermore found that the glycosylation of the thrombomodulin is responsible for accelerating the anticoagulatory effect of antithrombin III and, as a result, the novel method determines another important anticoagulatory mechanism, i.e. antithrombin III itself, in addition to the protein C system. Consequently, this document describes, for the first time, a method which determines both important mechanisms for regulating coagulation.

The cause of this surprising effect is possibly not so much the inhibition of fibrinogen cleavage which is associated with the inhibition of thrombin but probably more likely the inhibition of factor V activation; an unlimited factor V activation would lead to a supply of thrombin which is increased by a factor of about 10,000, which thrombin can then no longer be so effectively captured by thrombomodulin.

Based on these new insights, a shortened coagulation time is to be expected in the presence of thrombomodulin when an antithrombin III-deficient plasma is used instead of a normal plasma, since antithrombin III can no longer neutralize the procoagulatory activities of the thrombin which is complexed with thrombomodulin. The prolongation of the coagulation time is also less pronounced, as compared with a normal plasma, when there is a defect or a disturbance in the protein C system. Consequently, defects in both systems act in the same direction. This was shown for the first time in Example 6.

Thrombomodulin which has an intact glycosylation has therefore to be used for describing the physiological function of the protein C system and of antithrombin III, i.e. the thrombomodulin which is used must possess both the thrombin-inhibiting activity (activity B) and the protein C-activating activity (activity A). Based on this insight, it is possible to determine the procoagulatory activity using a thromboplastin-containing reagent since this represents the physiologically relevant, extrinsic coagulation pathway and there is no need to preincubate the sample in order to activate the contact phase (Example 3).

In order to develop a method which is based on thromboplastin, the concentration of the thromboplastin has to be chosen such that the production of thrombin proceeds so slowly that, during this period, sufficient activated protein C is formed to retard the production of thrombin which is required for clot formation. For this, the skilled person adjusts the thromboplastin concentration in a reagent such that the coagulation time of a normal plasma in the absence of thrombomodulin is at least 20 s and at most 300 s, preferably in the range from 40 to 150 s. This can be achieved, for example, by diluting commercial thromboplastin reagents. A solution which contains the calcium ions which are required for the coagulation activity is preferably used for the dilution.

In addition, phospholipids, in suitable quantity (from 0.001 to 1% w/v) and nature (preferably from tissue extracts, such as thrombocytes, lung, placenta or brain, or from vegetable sources), should also be substituted when diluting the reagent. These sources usually contain sufficiently high proportions of phosphatidylethanolamine, a phospholipid which is important for the activity of activated protein C. However this compound can also be metered in, as required, in order to stimulate the A activity of the thrombomodulin and the protein C system.

In order to determine the optimum combination of thromboplastin concentration and thrombomodulin concentration, a curve family is constructed in which the coagulation time, with or without a particular concentration of thrombomodulin, is determined in relation to the dilution of the thromboplastin, with this determination being repeated at different thrombomodulin concentrations (see Example 4).

Thrombomodulin concentrations of between 0.5 and 50 μg/ml, based on the final volume of the test assay, are preferably employed, particularly preferably concentrations of between 1 and 10 μg/ml. The following combinations from this curve family are found to be suitable: those which, in the presence of thrombomodulin, exhibit coagulation times with a normal plasma which are less than 300 sec, particularly preferably less than 150 s, and in which the difference in relation to the coagulation time without thrombomodulin is at least 50%, preferably 100-300% of this coagulation time without thrombomodulin.

The thromboplastin can be derived from natural sources, such as placenta, lung or brain of human or animal origin, and can also have been produced by recombinant means.

The thrombomodulin is preferably isolated, using methods which are known per se to the skilled person, from natural sources, such as placenta, lung or brain of human or animal origin. A characteristic feature of the thrombomodulin is that the thrombomodulin-containing fractions exhibit an anti-thrombin effect which is augmented by antithrombin III, in addition to exhibiting the activation of protein C.

It is also known to prepare thrombomodulin recombinantly. The activity B has to be added post-translationally to the unglycosylated, recombinant thrombomodulin by coupling the thrombomodulin bio-chemically or chemically to a heparin sulfate. This can also be achieved by expressing the thrombomodulin in glycosylating cells, e.g. cells of human origin. It was found, surprisingly, that the requisite effect is also achieved by adding heparin sulfate which is not bound to thrombomodulin (Example 7).

Known aggregation inhibitors, such as fibrin cleavage products which are obtained by cleaving fibrinogen with cyanogen bromide, plasmin, elastase or other known enzymes, for example from snake venoms (see, for example, Markland F S Jr., Thromb. Haemostas. 1991; 65: 438-443), or synthetic peptides which possess the RGD sequence, as described in EP-A-0 456 152, for example, can be added to the reagent in order to avoid premature clot formation.

Substances which are known per se to the skilled person, such as potassium hexacyanoferrate, vitamin C, glutathione, uric acid, hydroquinone, tocopherols, butylhydroxytoluene (BHT), butylhydroxyaniline, ubiquinone or enzymes, such as superoxide dismutase and catalase, can be used for oxidation protection in order to rule out oxidation of the thrombomodulin or the phospholipids in the reagent.

Based on this novel method, individual parts of the anticoagulatory system can be visualized by making additions to the reagents or the samples. Thus, antithrombin III can be added, for example, so that only disturbances of the protein C system are detected. Conversely, the sample can be mixed, for example, with an antithrombin III-deficient plasma in order to cut out disturbances of the protein C system. In addition, plasmas which do not contain one or more factors of the protein C system, whether this is because the plasmas are natural, for example congenital deficient plasmas, or because these factors have been removed using a technique, for example immunoadsorption, can be used in order to be added to the sample plasma such that the only disturbances of factors to become apparent are those which do not exist in the plasma which is used for the mixing. Phospholipids, for example from thrombocytes, can also be added to reagents, or directly to the sample and/or deficient plasmas, in order to neutralize the effect of anti-phospholipid antibodies, for example lupus anticoagulants.

The observation that the anticoagulatory effect (activity B) of thrombomodulin is required in order to produce a plainly recognizable retardation of coagulation activity by way of the protein C system leads to a novel interpretation with regard to the biological importance of the carbohydrate moiety and diagnostic use. The true biological significance of the carbohydrate moiety of glycoproteins is so far unknown. As a rule, discussion is only centered around a possible influence on half-life in the circulation (Paulson J C, TIBS 1989; 14: 272-275). However, it is known that diabetics exhibit a relatively high incidence of thromboses, especially in the arterial vascular system. Since, on the basis of the conclusions which have been presented here, the anticoagulatory effect on thrombin is the prerequisite for the anticoagulatory activity of protein C, and, on the other hand, a disturbance in the correct synthesis of the carbohydrate moiety can occur in diabetics, it is presumed that the loss of the anti-thrombin activity of thrombomodulin in diabetics plays an important role in the pathological mechanism which leads to an increased risk of thrombosis.

This means that detection of the glycosylation or activity A (anti-thrombin effect) of thrombomodulin in relation to activity B (protein C activation) represents an important diagnostic marker for anticoagulatory protein C potential on the vascular surface, and can consequently be used for assessing the risk of thrombosis, in particular arterial thromboses. This analysis is preferably carried out in diabetics or persons suffering from a disturbance in methionine metabolism (hyperhomocysteinemia) in order to determine the progress of the damage to the endothelium. This analysis can also provide important prognostic or therapeutically meaningful information in the case of other diseases, such as tumors, atherosclerosis, autoimmune diseases or other inflammatory diseases, which are associated with a disturbance in the metabolism of the endothelium.

The ratio of the two activities can be detected both using cleavage products, of thrombomodulin which occur naturally in the plasma and by means of analyzing the natural tissue of patients. The two activities are determined separately in chromogenic tests, as described, for example, in Preissner et al. (J. Biol. Chem. 1990; 265: 4915-4922; see Example 1 as well). The thrombomodulin is preferably separated from the remaining matrix (for example plasma constituents) before the determination takes place. A test kit which comprises a solid phase which is coated with antibodies against thrombomodulin, for example a microtiter plate, test strip or test module, is suitable for this purpose. In a first incubation step, the thrombomodulin is bound to the solid phase and interfering matrix is then removed by washing. After that, the proportions of the two activities are determined chromogenically in separate test assays.

Furthermore, the degree to which the thrombomodulin isolated from the blood or tissue of patients is glycosylated can be determined directly using methods which are known to the skilled person. The results, for example the ratio of activity A to activity B, or vice versa, serve as a measure of the severity of the disturbance in synthesis and/or as an indicator of an increased risk of thrombosis.

### EXAMPLES

The following examples are only intended to illustrate the invention and not to limit it. Unless otherwise indicated, the reagents and equipment used were from Behring Diagnostics GmbH.

### Example 1

Chromogenic Determination of Thrombomodulin Activity A (Protein C Activation)

The chromogenic determination of the thrombomodulin activity is based on Salem et al., Journal of Biological Chemistry, Vol. 259, No. 19, pp. 12246-12251 (1984). The test was carried out using a Behring Coagulation Timer (Behring Diagnostics):

50 μl of sample were mixed with 50 μl of reagent 1, which comprised 50 μg of protein C/ml and 6 μg of thrombin/ml in 50 mM Tris-HCl, 200 mM NaCl, 5 mM MnCl₂, 1% bovine serum albumin, pH 7.3, and the whole was incubated for 5 minutes. During this time, thrombin, acting together with the thrombomodulin which is present in the sample, activates protein C to form activated protein C. This activation is interrupted by adding an inhibitor cocktail (50 U of antithrombin III/ml, 5 antithrombin units of hirudin/ml and 1 U of unfractionated heparin/ml in 50 mM Tris-HCl, 100 mM NaCl, 5 mM EDTA, pH 7.4) and, after 30 seconds, 50 μl of a chromogenic protein C substrate (composed of Berichrom protein C; Behring Diagnostics) are added. The color development is monitored at 405 nm for 30 seconds, and the change in extinction per minute (delta U/min) is calculated from this. This change is proportional to the quantity of thrombomodulin in the sample. The values listed in Table 1 were obtained with rabbit thrombomodulin (from American Diagnostics; 1000 U/mg of protein).

TABLE 1
Determination of the thrombomodulin activity in the
chromogenic test.
(TM deglyc = chondroitinase-treated thrombomodulin; see
Example 2)

| Thrombomodulin | |
|---|---|
| (μg/ml) | Delta U/min* 10⁻³ |
| 0 | 20.0 |
| 0.2 | 53.9 |
| 0.4 | 81.0 |
| 0.6 | 106.0 |
| 0.8 | 155.1 |
| 1.0 | 169.0 |
| 1.5 | 210.7 |
| 2.0 | 247.3 |
| TM deglyc. | Delta U/min* 10⁻³ |
| (μg/ml) | |
| 0.5 | 113.4 |

### Example 2

Removal of the Carbohydrate Moiety of the Thrombomodulin

For the purpose of removing the heparin moiety of the thrombomodulin, 30 μl of a chondroitinase ABC solution (10 U/ml; from Sigma) were added to 1 ml containing 30 μg of rabbit thrombomodulin (from American Diagnostica)/ml in 50 mM Tris-HCl, pH 8.0, and the mixture was incubated at +37° C. overnight.

The treated thrombomodulin was diluted to 0.5 μg/ml in reagent buffer 1 from Example 1, and tested. At 0.5 μg/ml, the pretreated thrombomodulin exhibited an activity which corresponds to 0.6 μg of the untreated thrombomodulin/ml. Consequently, the protein C-activating activity was not reduced but, on the contrary, slightly increased.

### Example 3

Prolonging the APTT by Adding Thrombomodulin

As described in Matschiner (U.S. Pat. No. 5,525,478), 50 μl of an APTT reagent (actin; from Dade) were added to 50 μl of a plasma pool from normal donors, and the mixture was incubated at +37° C. for 120 sec in order to activate the contact phase. 50 μl of thrombomodulin (from rabbit; 15 μg/ml in physiological sodium chloride solution) were added instead of the calcium chloride which is otherwise added in the case of an APTT, and only after that was coagulation triggered by adding calcium chloride (25 mmol/l). The results are listed in Table 2. They show, first of all, that the coagulation times are prolonged, as compared with a normal sample, when thrombomodulin is present. This is in agreement with Matschiner's data.

However, a similar, even if smaller prolongation is also seen in the case of the protein C-deficient plasma. In this case, we assume that this can be attributed to the inhibitory effect (activity B) of the thrombomodulin on thrombin rather than to inactivation of the procoagulatory cofactors VIIIa and Va (activity A). This becomes clear when, as in this example, use is made of thrombomodulin whose glycosylation has been removed. Surprisingly, it is now not only the coagulation prolongation due to activity B which is eliminated; the coagulation prolongation due to activity A is also almost completely eliminated (approx. 20 sec; =difference between protein C-deficient plasma with and without thrombomodulin).

This has not been shown previously. It leads to the conclusion that activity B is a prerequisite for activity A under physiological conditions (fibrin formation) and, as a consequence, only thrombomodulin which possesses an intact glycosylation can be used in the coagulation test.

Table 2

Influence of thrombomodulin (3.75 μg/ml in the test assay) on the APTT of normal plasma and protein C-deficient plasma. The values given are the coagulation times in sec.

TM = thrombomodulin. Intact = natural, glycosylated
thrombomodulin; deglyc = following treatment with
chondrotinase ABC (see Example 2).

| | | Protein C-deficient |
|---|---|---|
| Addition of TM | Normal plasma | plasma |
| No addition | 28.2 | 32.7 |
| Intact TM | 166.4 | 63.2 |
| Deglyc TM | 40.4 | 44.6 |

### Example 4

Prolongation of the PT of a Normal Plasma by Thrombomodulin in Dependence on the Concentration of Thromboplastin and Thrombomodulin

In the novel method, a suitable concentration of a thromboplastin—containing PT reagent has to be sought, for a given concentration of thrombomodulin, in order to achieve a prolongation of the coagulation time (20-300 sec) which is suitable for a test for screening the protein C system.

For this, 1 part of a thrombomodulin-containing reagent (rabbit thrombomodulin in 50 mM Tris-HCl, with or without 0.025% soybean phospholipid, pH 7.4) was added to 1 part of sample, and the coagulation reaction was triggered with different dilutions of a PT reagent (in this case, by way of example, Thromborel S, Behring Diagnostics; dilution with 25 mM calcium chloride solution). For comparison, the coagulation time was determined without adding thrombomodulin.

The example recorded in Table 3 shows that, at a low concentration of thrombomodulin, the coagulation times initially remain the same as those for a PT without thrombomodulin as the dilution of the thromboplastin increases, and a difference is only obtained at high dilution (in this case: 1:1000). If, on the other hand, a higher concentration of thrombomodulin is chosen, a difference can already be seen at lower dilution. In this experiment, an optimum combination would be selected at a thromboplastin dilution of 1:100 in the presence of 1.7 μg of thrombomodulin/ml (in the test assay) (see Example 5 as well) since the differences are too low at lower dilutions of thromboplastin. At the 1:100 dilution, the difference in the presence of 1.4 μg/ml is somewhat low, while in the presence of 2.0 μg/ml it is somewhat high. As a consequence, this method requires markedly less thrombomodulin in the test assay than does the Matschiner method and is superior to the latter in this respect.

At higher dilution, longer coagulation times with and without TM are obtained when phospholipids are not substituted as compared with when phospholipids are substituted (see Table 4). When phospholipids are not substituted, the difference between the control test without thrombomodulin and the actual screening test with thrombomodulin is somewhat poorer. When phospholipids are substituted, the fact must be taken into account that this reduces the sensitivity to lupus anticoagulants in the method.

TABLE 3
Influence of the thromboplastin concentration on the PT
of a normal plasma in the absence and the presence of
different concentrations of thrombomodulin at a constant
concentration of phospholipid. The values given are the
coagulation times in sec and the differences as compared
with the coagulation time without thrombomodulin in the
assay. TM = thrombomodulin; PL = phospholipids;
Diff. = difference between coagulation time with and
without TM;

| | 0 | 0.7 | | 1.4 | | 2.0 | |
|---|---|---|---|---|---|---|---|
| Dilution | μg/ml | μg/ml | Diff. | μg/ml | Diff. | μg/ml | Diff. |
| 1:10 | 22.9 | 24.0 | 1.1 | 24.6 | 1.7 | 25.7 | 2.8 |
| 1:30 | 31.5 | 35.2 | 3.7 | 35.6 | 4.1 | 41.9 | 10.4 |
| 1:100 | 44.7 | 53.9 | 9.2 | 83.9 | 39.2 | 231.8 | 187.1 |
| 1:300 | 62.7 | 101 | 38.3 | >300 | | >300 | |
| 1:1000 | 103.9 | >300 | | >300 | | >300 | |

TABLE 4
Influence of the thromboplastin concentration on the PT
of a normal plasma in the absence and the presence of
different concentrations of phospholipids at constant
thrombomodulin concentration (0.7 μg/ml). The values
given are the coagulation times in sec and the
difference between the coagulation time with and without
thrombomodulin in the assay. TM = thrombomodulin; PL =
phospholipids

| | without | with | | without | with | |
|---|---|---|---|---|---|---|
| | TM | TM | Diff. | TM | TM | Diff. |
| Dilu- | with | with | with | without | without | without |
| tion | PL | PL | PL | PL | PL | PL |
| 1:10 | 22.9 | 24.0 | 1.1 | 22.5 | 23.7 | 1.2 |
| 1:30 | 31.5 | 35.2 | 3.7 | 33.5 | 37.8 | 4.3 |
| 1:100 | 44.7 | 53.9 | 9.2 | 54.8 | 65.4 | 10.6 |
| 1:300 | 62.7 | 101 | 38.3 | 84.9 | 109.6 | 24.7 |
| 1:1000 | 103.9 | >300 | >200 | 154.2 | 281.9 | 127.7 |

### Example 5

Behavior, in the Novel Method, of Plasmas with Defects in the Anticoagulatory System

An optimum combination of activator reagent (in this case: thromboplastin) and thrombomodulin was determined from the curve families which were determined as described in Example 4. Table 5 shows, for such a combination, the reaction of different plasmas having defects in the protein C system. Thromborel S was diluted 1:100 with calcium chloride, as in Example 4. The thrombomodulin reagent contained 5.0 μg of thrombomodulin/ml (corresponds to 1.7 μg/ml in the test assay) and also phospholipids (0.025%). The following plasmas were tested: a normal plasma pool, a protein C-deficient plasma, a protein S-deficient plasma and a factor V disease plasma. The results in Table 5 provide evidence that, under these conditions, the coagulation times of the pathological samples were shortened as compared with the normal plasma in the presence of thrombomodulin.

This shows that a test based on a dilute PT in the presence of thrombomodulin indicates disturbances of the protein C system by means of a less pronounced prolongation of the coagulation time.

TABLE 5
Coagulation times (in sec) of different plasmas with
defects in the protein C system as compared with a
normal plasma pool in the novel method.
TM = thrombomodulin

| | | | Difference | Ratio |
|---|---|---|---|---|
| | | | (with- | (with/ |
| | without | with | without) | without) |
| Sample | TM | TM | TM | TM |
| Normal plasma | 34.3 | 125.6 | 91.3 | 3.7 |
| Protein C-DP | 37.4 | 62.9 | 25.5 | 1.7 |
| Protein S-DP | 41.3 | 68.4 | 27.1 | 1.7 |
| Factor V disease | 39.8 | 103.6 | 63.8 | 2.6 |
| (heterozygous | | | | |
| defect) | | | | |

### Example 6

Behavior, in the Novel Method, of an Antithrombin III-deficient Plasma

A plasma with a congenital antithrombin III defect (<0.01 U of AT III/ml; from Milan Analytica AG, Switzerland), but with other coagulation factors in the normal range, was used in the novel method as described in Example 5. Differently from Example 5, the thrombomodulin reagent contained 7.0 μg of thrombomodulin/ml (corresponds to 2.3 μg/ml in the test assay) in order to achieve an optimum reaction, as shown in Example 4. The same normal plasma pool as in Example 5 was included for comparison.

The results shown in Table 6 provide evidence that the coagulation time of the antithrombin-deficient plasma was shortened as compared with the normal plasma in the presence of thrombomodulin. This shortening indicates that the anticoagulation potential is incomplete as compared with a normal plasma. Consequently, the novel method makes it possible, for the first time, to detect defects in both the important anticoagulatory systems: i.e. the protein C system and the antithrombin III system.

TABLE 6
Coagulation times (in sec) of a plasma with an anti-
thrombin III deficiency (<0.001 U/ml), as compared with
a normal plasma pool, in the novel method. The values
given are the coagulation times with and without
thrombomodulin, and also the difference between and the
ratio of the two coagulation times. TM = thrombomodulin

| | | | Difference | Ratio |
|---|---|---|---|---|
| | | | (with- | (with/ |
| | without | with | without) | without) |
| Sample | TM | TM | TM | TM |
| Normal plasma | 37.4 | 128.5 | 91.1 | 3.4 |
| Antithrombin III DP | 42.7 | 79.9 | 37.2 | 1.9 |

### Example 7

Substituting for the Natural Glycosylation by Adding a Heparin Sulfate to a Deglycosylated Thrombomodulin.

The rabbit thrombomodulin was deglycosylated (TM deglyc) by using chondroitinase, as described in Example 2.

As in Example 4, thromborel S was diluted 1:100 with 25 mmol/l calcium chloride. The thrombomodulin reagent contained 10 μg of thrombomodulin/ml (corresponds to 3.3 μg/ml in the test assay) and soybean phospholipids (0.05%). Due to the deglycosylation, only a very small prolongation of the coagulation time is achieved in the novel method, as is shown for a normal plasma pool in Table 7. When heparin is added to the thrombomodulin-containing reagent (1 U/ml; corresponds to 0.33 U/ml in the test assay; Liquemin, from Hoffmann LaRoche, Switzerland), the coagulation time in the absence of thrombomodulin is also prolonged due to inhibition of the procoagulatory reaction; in the presence of thrombomodulin, on the other hand, the coagulation time is prolonged several fold. This is to be attributed to the anticoagulatory property of the thrombomodulin having been restored by the addition of heparin since, when a plasma having a defect in the protein C system (heterozygous factor V disease defect plasma) is used, the difference or ratio between these two coagulation times is much less pronounced.

The novel method can, therefore, also be carried out using unglycosylated thrombomodulin, for example recombinantly prepared thrombomodulin, by adding heparin to the test assay.

TABLE 7
Coagulation times (in sec) of normal plasma and a plasma
with a defect in the protein C system (heterozygous
factor V disease defect) when using glycosylated
thrombomodulin (gly) or deglycosylated thrombomodulin
(degly), without (—) or with the addition (hep) of 1 U
of heparin/ml in the novel method. The values given are
the coagulation times with and without thrombomodulin,
and the difference between and the ratio of the two
coagulation times. TM = thrombomodulin

| | TM/ | without | with | Differ- | |
|---|---|---|---|---|---|
| Sample | heparin | TM | TM | ence | Ratio |
| Normal plasma | gly | 32.3 | 106.8 | 74.5 | 3.3 |
| | degly/— | 35.0 | 45.7 | 10.7 | 1.3 |
| | degly/hep | 65.9 | 189.5 | 123.6 | 2.9 |
| Factor V disease | degly/hep | 61.9 | 99.3 | 37.4 | 1.6 |

1. A method for determining the anticoagulatory potential of a sample in the presence of exogenously added thrombomodulin, which method includes the following steps:
a) the following reagents are added to the sample, preferably a plasma sample:
i) exogenous thrombomodulin which can form a complex with thrombin, with this complex being able to activate the protein C in the sample, and with it being possible for the protein C to be endogenous protein C or exogenously added protein C,
ii) an activator which leads, without any further intermediate incubation, to the activation of thrombin, with it being possible for the prothrombin to be endogenous prothrombin or exogenously added prothrombin,
iii) phospholipids,
iv) calcium ions,
v) and also other additional reagents which are used generally for optimizing coagulation tests,
b) the reaction is started by adding the prothrombin activator-containing reagent, and
c) the formation of thrombin is determined by measuring the transformation rate of a thrombin substrate, with this transformation rate being determined by measuring the time until a fibrin clot has formed or by the transformation rate of a labeled thrombin substrate.

2. The method as claimed in claim 1, wherein the measured transformation rate is related to the transformation rate in a test assay for determining coagulation time in which no activated protein C is formed or added.

3. The method as claimed in claim 2, wherein the measured transformation rate is related to the transformation rate in a test assay which is analogous to the method as claimed in claim 1, but in which no thrombomodulin is added.

4. The method as claimed in at least one of claims 1 to 3, wherein use is made of a thrombomodulin which, in addition to its protein C-activating activity (PCaA) also possesses the property of accelerating the inhibition of thrombin by antithrombin III (AITA).

5. The method as claimed in at least one of claims 1 to 4, wherein the concentration of the activator in step a) ii) is chosen such that the coagulation time of a normal plasma in the absence of thrombomodulin is at least 20 s and at most 300 s, preferably from 30 to 150 s.

6. The method as claimed in at least one of claims 1 to 5, wherein use is made, as activators in step a) ii), of thromboplastin reagents which are known per se to the skilled person and which are of natural human or animal origin, such as from placenta, lung or brain, or are produced by recombinant means.

7. The method as claimed in claim 1, wherein the thrombomodulin is added to the sample in a separate reagent, separately from the activator-containing reagent.

8. The method as claimed in claim 1, wherein the thrombomodulin employed may be of human, animal, recombinant or synthetic origin, preferably of human origin or from rabbit, particularly preferably from rabbit.

9. The method as claimed in claim 8, wherein, in the case of recombinantly prepared thrombomodulin, the thrombin-inactivating property is restored by linking to a glycosaminoglycan, preferably heparin sulfate.

10. The method as claimed in claim 8, wherein, in the case of recombinantly prepared thrombomodulin, the thrombin-inactivating property is restored by adding a glycosaminoglycan, preferably heparin sulfate.

11. The method as claimed in claim 9, wherein the linking is effected recombinantly or synthetically.

12. The method as claimed in at least claim 1, wherein the quantity of thrombomodulin in the reagent is selected such that, in the presence of thrombomodulin, the coagulation times with a normal plasma are less than 300 sec, particularly preferably less than 150 sec, and wherein the difference in relation to the coagulation time without thrombomodulin is at least 40%, preferably 100 to 300% of this coagulation time without thrombomodulin.

13. The method as claimed in at least one of claims 1 to 12, wherein the thrombomodulin concentration is from 0.5 to 50 μg/ml, preferably from 1 to 10 μg/ml, based on the final volume of the test assay.

14. The method as claimed in at least one of claims 1 to 13, wherein known aggregation inhibitors are added in the test procedure in order to retard clot formation.

15. The method, as claimed in one of claims 1 to 14, wherein purified coagulation factors which are not involved in the function of the protein C system or of the antithrombin III system are substituted by addition to the reagent or reagents.

16. The method as claimed in claim 15, wherein fibrinogen, factor VII, factor IX, factor X and/or prothrombin (factor II) are added to the reagent or reagents at concentrations such that, based on the sample, concentrations of 50-200%, preferably of from 70 to 150%, are reached.

17. The method as claimed in claim 15, wherein, in order to exclude an antithrombin III deficiency or defect in the sample, antithrombin III is present in one or more reagents in such a quantity that, based on the quantity of sample, concentrations of 50-200%, preferably of from 70 to 150%, are reached.

18. The method as claimed in claim 1, wherein, in order to selectively determine single or multiple disturbances, a solution which contains coagulation factors which are not to be codetected in the test is added to the plasma sample before it is used in the method.

19. The method as claimed in claim 18, wherein, in order to selectively diagnose the defect in or lack of a protein, the sample is prediluted, before being used in the method, in a ratio of from 1:2 to 1:20, preferably of from 1:3 to 1:5, particularly preferably of 1:4, with a plasma which contains less than 5% of this protein.

20. The method as claimed in claim 18, wherein, in order to selectively diagnose a defect in or lack of several proteins, the sample is prediluted, before being used in the method, in a ratio of from 1:2 to 1:20, preferably of from 1:3 to 1:5, particularly preferably of 1:4, with a plasma which contains less than 5% of each of these proteins.

21. The method as claimed in claim 18, wherein, in order to selectively diagnose anti-phospholipid antibodies, the sample is prediluted, before being used in the method, in a ratio of from 1:2 to 1:20, preferably of from 1:3 to 1:5, particularly preferably of 1:4, with an aqueous solution which contains phospholipids and/or thrombocytes at a concentration of from 0.01 to 1%.

22. The method as claimed in claim 18, wherein, in order to determine the anticoagulatory activity of antithrombin III, the sample is prediluted, in a ratio of from 1:2 to 1:10, preferably of 1:4, with an antithrombin III-deficient plasma.

23. The method as claimed in claim 1, wherein the substrate transformation rate of the sample is determined in the presence of thrombomodulin and in the absence of thrombomodulin, and this difference, or the quotient of the two values, is related to the difference or the quotient which is obtained with a normal plasma or plasma pool.

24. The use of the method as claimed in claim 1 for identifying patients who are at an increased risk of thrombosis.

25. The use of the method as claimed in claim 1 for monitoring an anticoagulation therapy.

26. The use of the method as claimed in claim 1 for detecting and quantifying the glycosylation of the thrombomodulin of a patient by determining the ratio of the protein C-activating activity (PCaA) and the activity bringing about acceleration of the inhibition of thrombin by antithrombin III (AITA) of the thromboplastin in a patient sample.

27. The use as claimed in claim 26, wherein the two activities of the thrombomodulin are determined directly in the sample.

28. The method as claimed in claim 26, wherein the endogenous thrombomodulin is isolated from the sample before the two activities are determined.

29. A test kit for use in a method as claimed in claim 26, which comprises
a) a test strip which is coated with antibodies against thrombomodulin, which strip is brought into contact with the sample,
b) a washing solution in which the incubated test strip is washed,
c) reagents for determining protein C activation
d) reagents for determining thrombin inactivation.

30. A series of reagents for determining protein C activation as claimed in claim 29, which comprises, in one reagent, thrombin, protein C and calcium chloride, in which the test strip is initially incubated in order to activate protein C, and a second reagent, which comprises antithrombin III, heparin and/or hirudin and a chromogenic protein C substrate for inactivating the thrombin and determining the quantity of protein C formed by determining the color intensity of the test strip.

31. A series of reagents for determining thrombin inactivation as claimed in claim 29, which comprises, in one reagent, antithrombin III, into which the test strip is introduced, after which thrombin is added and, after an incubation period, the remaining thrombin activity is determined, by means of determining the color intensity of the test strip, by adding a chromogenic thrombin substrate.

32. A test kit as claimed in at least one of claims 26 to 31, wherein a microtiter plate coated with antibodies against thrombomodulin is used instead of a test strip.

33. The use of the method as claimed in claim 26 for determining the degree of glycosylation of thrombomodulin in blood, plasma or tissue from patients with diabetes or homocysteinemia in order to assess the severity of the disease and/or the thrombophilia.

34. The use of the method as claimed in claim 26 for determining the degree of glycosylation of thrombomodulin in blood, plasma or tissue from patients with atherosclerosis in order to assess the severity of the disease and/or the thrombophilia.

35. A method for determining the AT III activity and the protein C system activity of a sample in the presence of exogenously added thrombomodulin, which method includes the following steps:
a) the following reagents are added to the sample, preferably a plasma sample:
i) exogenous thrombomodulin which, in addition to its protein C-activating activity (PCaA), also possesses the property of accelerating the inhibition of thrombin by antithrombin III (AITA), or to which heparin is added in order to reconstitute the AITA property,
ii) at least one activator which leads, without any further intermediate incubation, to the activation of prothrombin to form thrombin, with it being possible for the prothrombin to be endogenous prothrombin or exogenously added prothrombin,
iii) phospholipids,
iv) calcium ions,
v) and also other additional reagents which are used generally for optimizing coagulation tests,
b) the formation of thrombin is determined by measuring the transformation rate of a thrombin substrate, with this transformation rate being determined by measuring the time until a fibrin clot has formed or by the transformation rate of a labeled thrombin substrate.

36. The method as claimed in claim 35 for selectively determining the AT III activity, wherein the sample is diluted, in a ratio of from 1:2 to 1:20, preferably of from 1:3 to 1:5, particularly preferably of 1:4, with a plasma which contains less than 5% of the normal AT III activity.
